(19) United States
(12) Patent Application Publication
(10) Pub. No.: US 2023/0241363 A1
(43) Pub. Date: Aug. 3, 2023

(54) SYSTEMS AND METHODS FOR A HYDROPHILIC CATHETER INTRODUCTION SYSTEM

(71) Applicant: Dignity Health, San Francisco, CA (US)

(72) Inventors: S. Harrison Farber, San Francisco, CA (US); Andrew Ducruet, San Francisco, CA (US)

(21) Appl. No.: 18/004,858

(22) PCT Filed: Jul. 9, 2021
(86) PCT No.: PCT/US2021/041043

§ 371 Date: Jan. 9, 2023

Related U.S. Application Data
(60) Provisional application No. 63/049,695, filed on Jul. 9, 2020.

(51) Int. Cl. A61M 27/00; A61M 25/00; A61M 25/01

(52) U.S. Cl. CPC A61M 27/006; A61M 25/0009; A61M 25/0111; A61M 2210/1017; A61M 2025/0681

(57) ABSTRACT

Various embodiments of a hydrophilic catheter introduction system for improved insertion of a shunt catheter are disclosed herein. The catheter introduction system includes an introducer sheath defining an inner surface that includes a hydrophilic coating to aid in passage of a catheter through the introducer sheath. Evaluation of the catheter introduction system with respect to other catheter introduction systems resulted in a significant decrease in average catheter threading time.

### FIELD

The present disclosure generally relates to medical devices, and in particular, to a system and associated method for a hydrophilic catheter introduction system for improved insertion of a ventriculoperitoneal shunt catheter.

### BACKGROUND

Ventriculoperitoneal shunt placement is a commonly performed neurosurgical procedure for draining excess cerebrospinal fluid from the brain to the peritoneum. A routine step in this procedure involves passing a catheter from the cranial incision to the abdominal incision in the subcutaneous plane. This step is performed by passing a metal passer underneath the skin inside a sheath to create a conduit for eventual passage of the catheter. The passer is then removed such that the sheath remains as a conduit through which the shunt catheter may be passed. In surgery, the shunt catheter often gets caught on the inside of the sheath, therefore making it difficult to thread the catheter from one incision to the other, particularly when the sheath is curved. Further, the position and angle of the catheter within the sheath is unclear when under the skin. Water may sometimes be injected from a distal end of the sheath to help facilitate passing the catheter. This can add time and difficulty to the procedure.

It is with these observations in mind, among others, that various aspects of the present disclosure were conceived and developed.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIG. 1 is an illustration showing a hydrophilic catheter introducer system including a sheath, an introducer and a catheter;

FIG. 2 is an illustration showing the sheath of FIG. 1;

FIG. 3 is an illustration showing the introducer inserted within the sheath of FIG. 1;

FIG. 4 is an illustration showing the catheter inserted within the sheath of FIG. 1;

FIG. 5 is an illustration showing placement of the sheath of FIG. 1 within a body;

FIG. 6 is a photograph showing an evaluation setup for evaluation of the sheath of FIG. 1;

FIG. 7 is a photograph showing a catheter becoming stuck within an uncoated sheath during the evaluation of FIG. 6;

FIGS. 8A and 8B are respective illustrations showing a catheter becoming stuck in an uncoated sheath and showing a catheter threaded through the sheath of FIG. 1; and

FIG. 9 is a graphical representation showing average threading time for uncoated sheaths and the sheath of FIG. 1.

Corresponding reference characters indicate corresponding elements among the view of the drawings. The headings used in the figures do not limit the scope of the claims.

### DETAILED DESCRIPTION

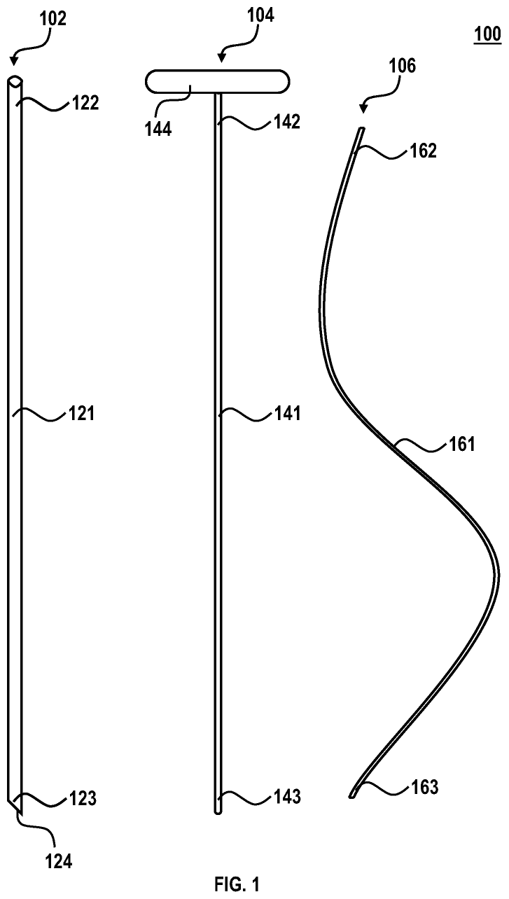

Various embodiments of a hydrophilic catheter introduction system for improved insertion of a shunt catheter are disclosed herein. In particular, the catheter introduction system includes a generally tubular sheath defining an inner lumen that defines an interior surface with a hydrophilic coating for improved threading of the shunt catheter through the sheath. The hydrophilic coating on the interior surface of the sheath reduces friction and prevents jamming of the shunt catheter while being passed through the inner lumen of the sheath. The sheath is configured for insertion within a body by a practitioner using an introducer coaxially positioned within the sheath. Upon successful insertion of the sheath within the body using the introducer, the practitioner can remove the introducer from the sheath, thereby leaving the sheath inside the body. The practitioner can then insert the shunt catheter into a proximal end of the sheath and feed the shunt catheter into the inner lumen of the sheath until the shunt catheter emerges from an opposite distal end of the sheath. Once the shunt catheter emerges, the practitioner can remove the sheath from the body. The hydrophilic coating along the interior surface of the sheath enables the shunt catheter to easily slide through the inner lumen of the sheath for improved passage of the shunt catheter from the proximal end of the sheath to the distal end of the sheath. In some embodiments, the catheter introduction system is configured for placement of a ventriculoperitoneal shunt catheter from the brain to the abdominal cavity. Referring to the drawings, embodiments of a hydrophilic catheter introduction system for improved insertion of a shunt catheter are illustrated and generally indicated as 100 in FIGS. 1-9.

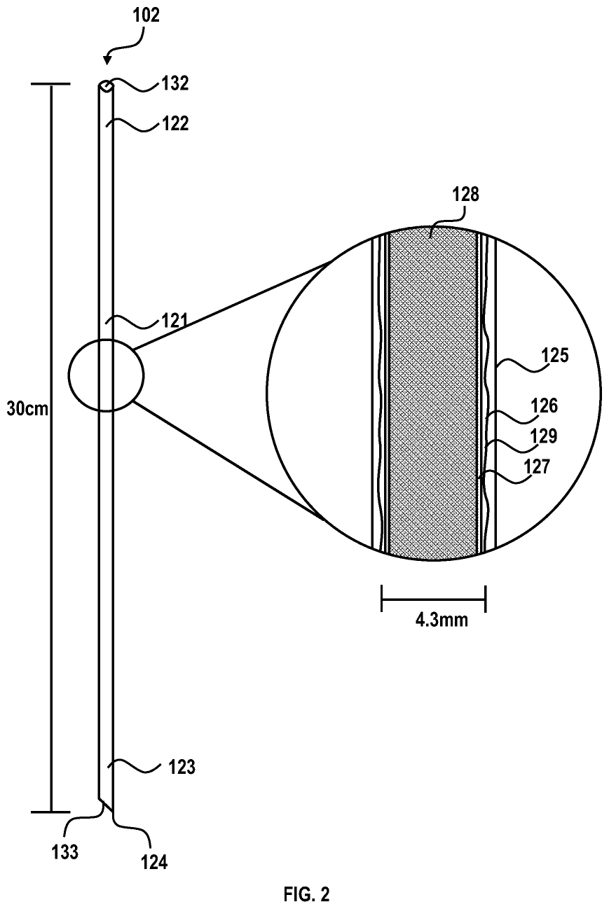

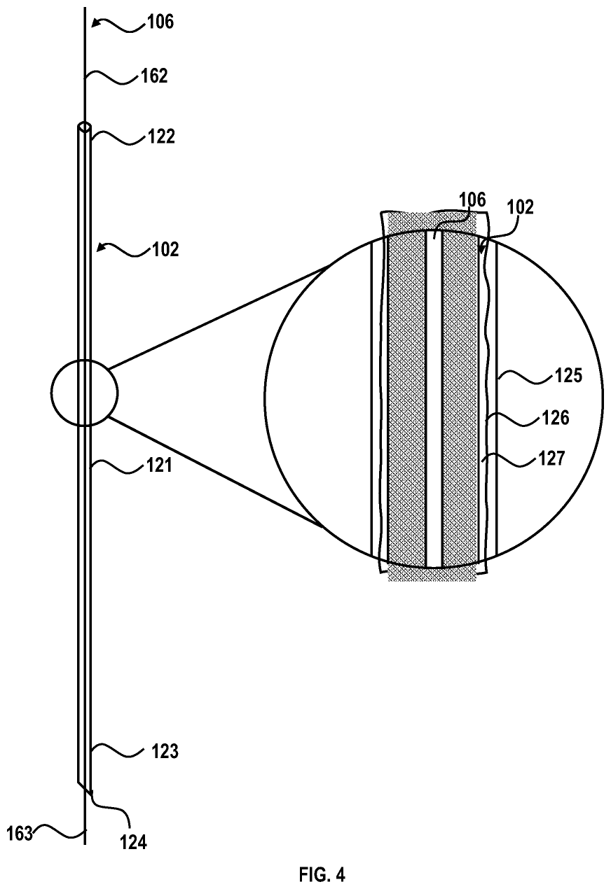

Referring to FIGS. 1-5 a hydrophilic catheter introduction system 100 is illustrated including a sheath 102. The sheath 102 forms a generally tubular sheath body 121 defining an interior surface 126 of an inner lumen that includes a layer of hydrophilic material 127 to reduce friction when threading a catheter 106 through the inner lumen of the sheath 102, as shown in FIGS. 2 and 4. The hydrophilic catheter introduction system 100 further includes an introducer 104 for introducing the sheath 102 into a body to create a channel within the body for eventual insertion of the catheter 106. Once the sheath 102 is inserted within the body using the introducer 104, the introducer 104 is removed and the catheter 106 is fed into the body through the sheath 102 until the catheter 106 emerges from the inner lumen at an opposite end of the sheath 102. The sheath 102 can then be removed from the body. The layer of hydrophilic material 127 reduces friction between the interior surface 126 of the inner lumen and the catheter 106 when inserting the catheter 106 into the sheath 102, thereby significantly reducing an amount of time it takes a practitioner to thread the catheter 106 through the sheath 102.

Referring to FIGS. 2, 5 and 8B, in some embodiments, the hydrophilic catheter introduction system 100 is configured for subcutaneous insertion of a ventriculoperitoneal catheter such as the catheter 106 for draining cerebrospinal fluid (CSF) from the brain and into the abdominal cavity. In many cases, an inserted sheath 102 can curve with the natural contours of the body, which commonly results in difficulty when threading the catheter 106 through the inner lumen of the sheath 102 as the catheter 106 tends to make contact with the interior surface 126 of the sheath 102 when being fed through curved portions of the sheath 102. The layer of hydrophilic material 127 enables the catheter 106 to easily slide along the interior surface 126 during insertion, rather than become caught when contacting the interior surface 126 of the sheath 102.

Referring to FIG. 2, the sheath 102 defines a generally tubular sheath body 121 that includes an outer surface 125 opposite to the interior surface 126 of the inner lumen 128 The sheath body 121 further defines a proximal portion 122 defining a proximal aperture 132 and an opposite distal portion 123 defining a distal aperture 133. The inner lumen 128 is defined through the sheath body 121 such that the proximal and distal portions 122 and 123 of the sheath 102 are in fluid flow communication with one another through the inner lumen 128. In particular, the proximal and distal apertures 131 and 132 of the proximal and distal portions 122 and 123 are in respective fluid flow communication with one another through the inner lumen 128. The inner lumen 128 is defined by the interior surface 126 of the sheath body 121 and is configured to receive the introducer 104 and the catheter 106. As shown in FIG. 2, the distal portion 123 of the sheath body 121 includes a tapered end 124 to aid in subcutaneous insertion of the sheath 102 into the body. In some embodiments, for insertion of a ventriculoperitoneal catheter, the length of the sheath 102 may be 30 cm and the internal diameter of the sheath body 121 may be 4.3 mm; however, other dimensions are contemplated for patients of varying sizes or for other placement locations within other parts of the body. In one aspect, a material of the sheath body 121 is a flexible nylon or other plastic such as polypropylene.

In one aspect, the layer of hydrophilic material 127 along the interior surface 126 of the sheath body 121 includes a properties that allow the interior surface 126 to become slippery in the presence of water for ease of insertion of the catheter 106 through the sheath 102. In some embodiments, the layer of hydrophilic material 127 includes one or more of the following hydrophilic materials: poly(vinylpyrrolidone), poly(ethylene oxide), poly(propylene oxide), polyacrylamide, methyl cellulose, polyacrylic acids, polyvinyl alcohols, and/or polyvinyl ethers, however any suitable hydrophilic coating that bonds to a flexible nylon or polymer surface such as the sheath body 121 can be utilized.

In some embodiments, the interior surface 126 is prepared with one or more layers of a base coat material 129 prior to coating the interior surface 126 with the layer of hydrophilic material 127. In another embodiment, the layer of hydrophilic material 127 can be grafted to the interior surface 126 of the sheath 102 by a graft copolymerization process.

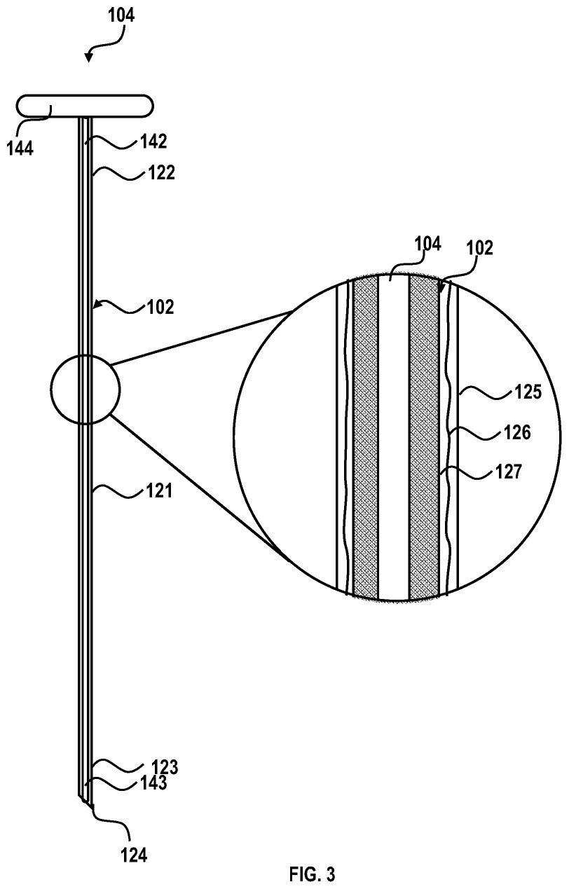

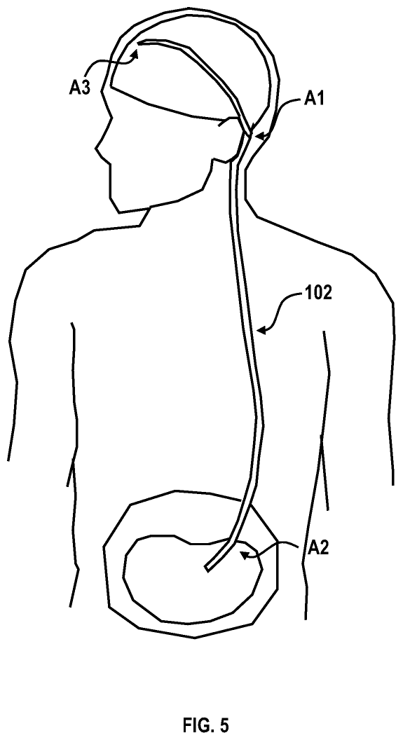

Referring to FIGS. 1, 3 and 5, in some embodiments the hydrophilic catheter introduction system 100 further includes the introducer 104 configured to aid in subcutaneous insertion of the sheath 102. In some embodiments, the introducer 104 includes an introducer body 141 defining an introducer proximal portion 142 and an opposite introducer distal portion 143. The introducer body 141 is at least semi-rigid to enable the introducer 104 and associated sheath 102 to penetrate and “tunnel” in the subcutaneous space along the body. In one particular embodiment shown in FIG. 5, the introducer body 141 is of an appropriate length and diameter to tunnel from a first incision A1 located near the brain to a second incision A2 located at the abdominal cavity. In particular, in the case of a ventriculoperitoneal catheter placement procedure, the second incision A2 is at the peritoneum. As further shown in FIGS. 1 and 3, the introducer proximal portion 142 includes a handle 144 that enables a practitioner to grip the introducer 104 and subcutaneously insert the introducer 104 and sheath into the body. The introducer body 141 is configured to be inserted in co-axial alignment into the lumen 128 of the sheath 102. In particular, the introducer distal portion 143 is first inserted into the proximal portion 122 of the sheath 102 until the introducer body 141 is fully inserted within the lumen 128 of the sheath 102 and the introducer distal portion 143 of the introducer body 141 is positioned at the distal portion 123 of the sheath 102. Once disposed inside the sheath 102, the introducer 104 and sheath 102 are inserted into the first incision A1 of the body. In the case of a ventriculoperitoneal catheter placement procedure, the first incision A1 is commonly placed posterior to the ear. The introducer 104 and sheath 102 are further pushed subcutaneously into the body until the second incision A2 is reached. Once the distal portion 123 of the sheath 102 is in an ideal position within and/or beyond the second incision A2, the introducer 104 is removed through the proximal portion 122 of the sheath 102. In some embodiments, the sheath 102 is inserted into a “frontal” area of the head with a third incision A3 behind the hairline. The sheath 102 is tunneled from the third incision A3 frontal area to the first incision A1 behind the ear and the catheter 106 is passed through. The sheath 102 is then removed and tunneling occurs from the first incision A1 behind the ear to the second incision A2 at the abdomen. In some embodiments, such as for insertion of a ventriculoperitoneal catheter, the length of the introducer 104 may be compatible with the length of the sheath 102, such as 30 cm, and the diameter of the introducer 104 may be less than the internal diameter of the sheath body 121; however other dimensions are contemplated for patients of varying sizes or for other placement locations within the body.

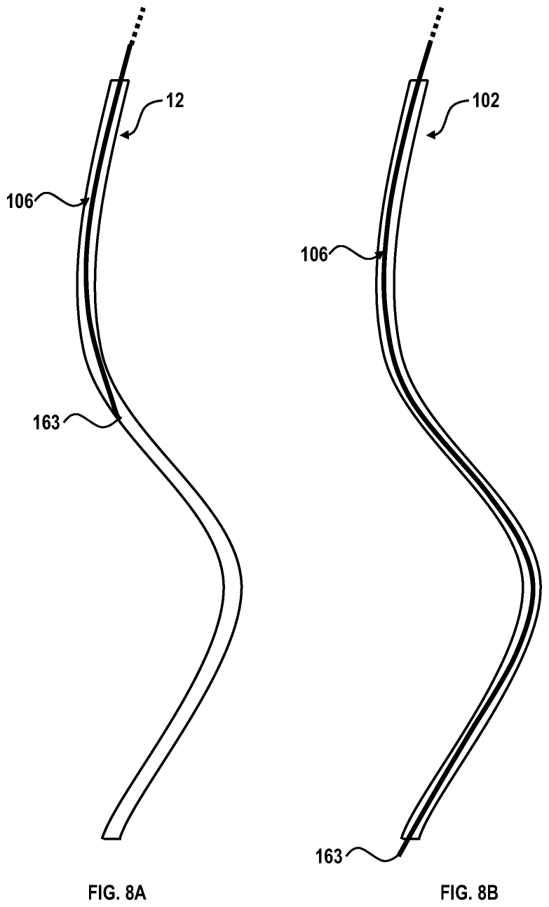

Referring to FIGS. 1 and 4, as noted above the hydrophilic catheter introduction system 100 further includes the catheter 106 configured for insertion within the body using the sheath 102. The catheter 106 defines a flexible tubular body 161 including a catheter proximal portion 162 and a catheter distal portion 163. As shown in FIGS. 4, 8A, and 8B, the catheter distal portion 163 of the catheter 106 is configured for insertion into the proximal aperture 132 of the sheath 102 and through the lumen 128 of the sheath 102 until the catheter distal portion 163 emerges from the distal aperture 133 of the sheath 102. The catheter distal portion 163 contacts the interior surface 126 of the sheath 102 coated with the layer of hydrophilic material 127 and easily slides along a direction of elongation of the sheath body 121. Upon successful placement of the catheter 106, the sheath 104 is removed with the catheter 106 remaining subcutaneously within the body.

In some embodiments, the sheath 102 of the hydrophilic catheter introduction system 100 having the layer of hydrophilic material 127 is manufactured with a bilaminar coating process. An uncoated sheath body 121 defining a lumen 128 having an interior surface 126 is provided. In some embodiments, at least one layer of base coat material 129 is applied to the interior surface 126 of the lumen 128. Following a curing process of the at least one layer of base coat material 129, a layer of hydrophilic material 127 is applied to the interior surface 126 of the inner lumen 128 over the layer of base coat material 129. The layer of hydrophilic material 127 is then subjected to a curing process such that the layer of hydrophilic material 127 bonds to the layer of base coat material. In some embodiments, the completed sheath 102 having the layer of hydrophilic material 127 is subjected to a sterilization process. In another embodiment, the sheath 102 of the hydrophilic catheter introduction system 100 having the layer of hydrophilic material 127 can be manufactured by a graft copolymerization process.

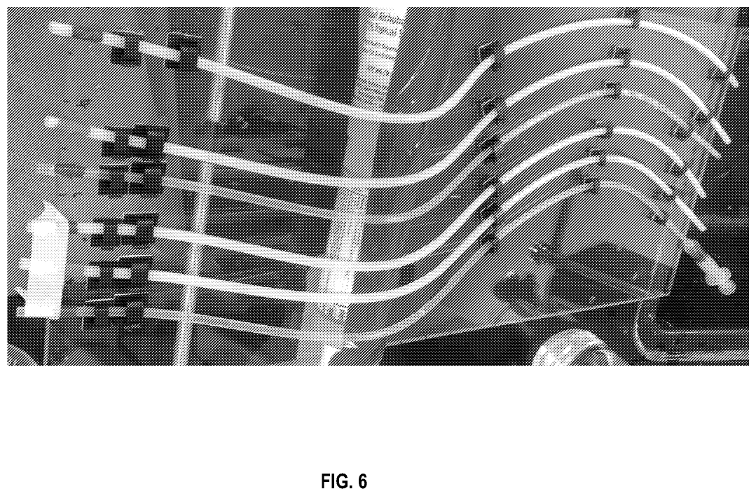

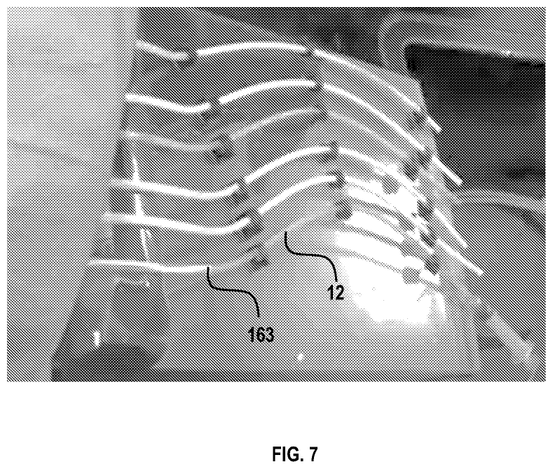

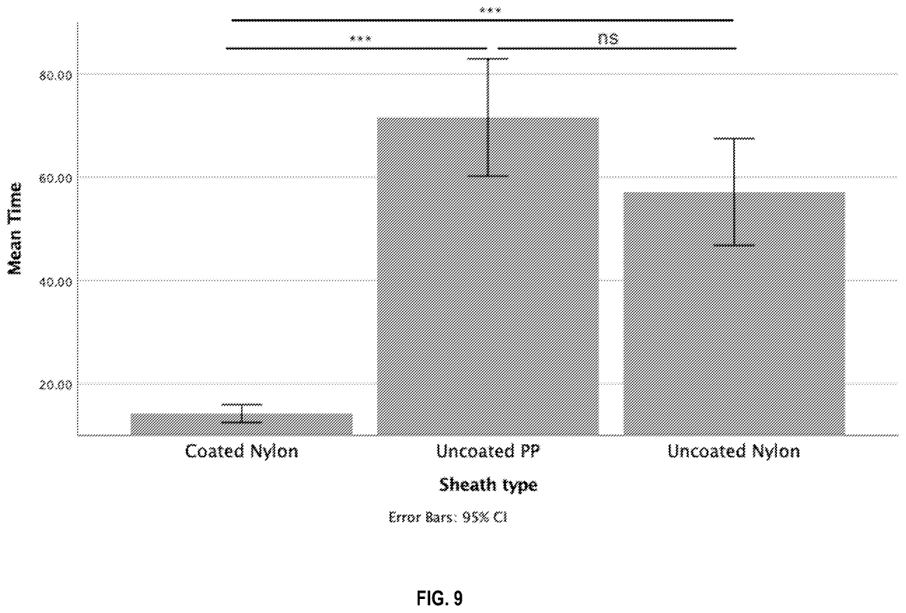

Referring to FIGS. 6-9, the hydrophilic catheter introduction system 100 was evaluated for efficacy. FIGS. 6 and 7 show an evaluation setup for the sheath 102 which involved threading a catheter 106 through the lumen of the sheath 102 having the layer of hydrophilic material 127 as well as threading a catheter 106 through a sheath 12 that did not include the layer of hydrophilic material 127. As illustrated in FIGS. 6-8B, the sheaths 102 and 12 were curved at multiple points to simulate a common curve of the body, as an inserted sheath such as sheath 102 would adhere to. FIGS. 7 and 8A illustrate a result of one such a test in which the catheter distal portion 163 became “stuck” on a curve within the uncoated sheath 12. However, as shown in FIG. 8B, the catheter 106 could be easily threaded through the sheath 102 having the layer of hydrophilic material 127 on the inner surface 126 of the sheath 102. FIG. 9 shows average (mean) time that it took practitioners to thread a catheter 106 through an uncoated polypropylene sheath, an uncoated nylon sheath, and a coated nylon sheath such as sheath 102. As illustrated, the coated nylon sheath 102 took under 20 seconds to thread, while the uncoated sheaths took over 50 seconds to thread. The layer of hydrophilic material decreases the frictional force required to pass the catheter 106 through the sheath 102 and ease this portion of a shunt procedure.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A sheath, comprising:
a sheath body defining an inner lumen having an interior surface; and
a layer of hydrophilic material coating along the interior surface of the inner lumen.

2. The sheath of claim 1, wherein the layer of hydrophilic material reduces an amount of friction between a distal portion of a catheter and the interior surface of the sheath body along the inner lumen.

3. The sheath of claim 1, wherein the sheath body comprises a flexible material.

4. The sheath of claim 3, wherein the flexible material includes polypropylene.

5. The sheath of claim 3, wherein the flexible material includes nylon.

6. The sheath of claim 1, wherein the sheath body is configured to receive a catheter.

7. The sheath of claim 1, wherein the sheath body defines a proximal portion and an opposite distal portion.

8. The sheath of claim 7, wherein the opposite distal portion of the sheath body defines a tapered end.

9. The sheath of claim 7, wherein the proximal portion of the sheath body is configured to receive a distal portion of a catheter.

10. The sheath of claim 7, wherein the proximal portion and the opposite distal portion of the sheath body are in fluid flow communication with one another by the inner lumen.

11. The sheath of claim 10, wherein the inner lumen is configured for passage of a catheter between the proximal portion and the opposite distal portion of the sheath body.

12. A method of manufacturing a hydrophilic sheath for a hydrophilic catheter introduction system, comprising:
providing a sheath body defining an inner lumen having an interior surface; and
applying a layer of hydrophilic material to the interior surface such that the layer of hydrophilic material bonds to the interior surface of the inner lumen.

13. The method of claim 12, further comprising:
applying a curing process to the hydrophilic coating.

14. The method of claim 12, further comprising:
applying a layer of base coat material.

15. The method of claim 14, further comprising:
applying a curing process to the base coat material.

16. The method of claim 12, further comprising:
sterilizing the sheath by a sterilization process.

17. The method of claim 12, further comprising:
bonding the hydrophilic layer to the interior surface of the lumen by a grafting process.

18. A sheath formed by the method of claim 12.
